(19) United States
(12) Patent Application Publication
(10) Pub. No.: US 2022/0287712 A1
(43) Pub. Date: Sep. 15, 2022

(54) SURGICAL CLIP APPLIER

(71) Applicant: Covidien LP, Mansfield, MA (US)

(72) Inventors: Jacob C. Baril, Norwalk, CT (US); Garrett P. Ebersole, Hamden, CT (US); Justin J. Thomas, New Haven, CT (US)

(21) Appl. No.: 17/572,669

(22) Filed: Jan. 11, 2022

Related U.S. Application Data
(60) Provisional application No. 63/158,457, filed on Mar. 9, 2021.

(51) Int. Cl. A61B 17/068; A61B 17/00

(52) U.S. Cl. CPC A61B 17/0682; A61B 2017/00327; A61B 2017/00296

(57) ABSTRACT

A surgical instrument, such as an endoscopic surgical clip applier, has a simplified handle assembly and a simplified drive assembly. The handle assembly and the drive assembly are configured to advance and form a surgical clip, for instance.

### CROSS-REFERENCE TO RELATED APPLICATION

This application claims the benefit of and priority to U.S. Provisional Patent Application No. 63/158,457, filed on Mar. 9, 2021, the entire disclosure of which is incorporated by reference herein.

### BACKGROUND

The disclosure relates to surgical instruments configured to apply surgical clips to tissue. More specifically, the disclosure relates to surgical clip appliers having a simplified handle assembly and a simplified drive assembly to advance and form surgical clips.

Surgical instruments, such as clip appliers, often include a handle assembly, an elongated member extending distally from the handle assembly, and an end effector engaged with a distal portion of the elongated member. The handle assembly typically include various components to facilitate the advancement of at least one drive member through the elongated member to affect a function of the end effector. The various components of the handle assembly may be challenging to assemble and/or complex to design.

### SUMMARY

The disclosure relates to an actuation mechanism for use with a surgical instrument having a housing, a pivotable handle, and a driver. The actuation mechanism includes a compliant member including a first arm, a second arm and a third arm. The first arm includes a first pivot point configured to pivotably engage the housing of the surgical instrument. The second arm includes a second pivot point configured to pivotably engage the pivotable handle of the surgical instrument. The third arm includes a third pivot point configured to pivotably engage the driver of the surgical instrument. The compliant member is a unitary structure, and is movable between a first position and a second position in response to the pivotable handle moving from a first, unactuated position to a second, actuated position.

In embodiments, the first arm and the second arm of the compliant member define a first angle therebetween, the second arm and the third arm of the compliant member define a second angle therebetween, and the first arm and the third arm of the compliant member define a third angle therebetween. The third angle is less than 180° when the compliant member is in its second position. It is also disclosed that the third angle is less than 180° when the compliant member is in its first position. It is further disclosed that the first angle and the second angle are obtuse when the compliant member is in the first position, and when the compliant member is in the second position.

In disclosed embodiments, the compliant member is made from one of a polymer, a composite material, a shape memory alloy, an elastomers, or a 3D-printed metal.

The disclosure also relates to a drive assembly for use with a surgical instrument, and includes a proximal driver, a driver connector, a distal driver, and a pusher assembly. The proximal driver is disposed in mechanical cooperation with a handle assembly of the surgical instrument. The driver connector includes a first leg and a second leg. The first leg is pivotably engaged with the proximal driver. The distal driver is pivotably engaged with the driver connector and is configured to form a surgical fastener. The pusher assembly includes a biasing element, a pusher pin, and a pusher rod. The biasing element biasing the pusher rod distally. Distal movement of the pusher rod is configured to distally advance a surgical fastener. The driver connector is movable between a first position where the driver connector is in contact with the pusher pin and prevents distal movement of the pusher pin, and a second position where the driver connector is free from contact with the pusher pin, thereby allowing distal movement of the pusher pin.

In embodiments, the driver assembly includes a slot. The pusher pin is configured to travel within the slot. It is disclosed that the slot is generally “U”-shaped.

In disclosed embodiments, the distal driver is pivotably engaged with the driver connector at an intersection between the first leg and the second leg.

It is further disclosed that distal movement of the proximal driver causes the driver connector to move from its first position to its second position, and that proximal movement of the proximal driver causes the driver connector to move from its second position to its first position.

The disclosure also relates to a surgical clip applier including a handle assembly, an elongated portion, an end effector, a driver assembly, and a compliant member. The handle assembly includes a housing, a pivotable handle, and a stationary handle. The elongated portion extends distally from the handle assembly and defines a longitudinal axis. The end effector is disposed adjacent a distal end of the end effector and includes a first jaw member and a second jaw member. The drive assembly is disposed at least partially within the elongated portion and includes a proximal driver, a driver connector, a distal driver, and a pusher assembly. The driver connector is pivotably engaged with the proximal driver and with the distal driver. The distal driver is configured to engage a portion of the end effector. The pusher assembly is configured to distally advance a surgical clip. The compliant member is disposed at least partially within the housing and includes a first arm, a second arm, and a third arm. The first arm includes a first pivot point configured to pivotably engage the housing of the handle assembly. The second arm includes a second pivot point configured to pivotably engage the pivotable handle of the handle assembly. The third arm includes a third pivot point configured to pivotably engage the proximal driver of the drive assembly.

In disclosed embodiments, the compliant member is a unitary structure, and is movable between a first position and a second position in response to the pivotable handle moving from a first, unactuated position to a second, actuated position. Additionally, it is disclosed that the first arm and the second arm of the compliant member define a first angle therebetween, the second arm and the third arm of the compliant member define a second angle therebetween, and the first arm and the third arm of the compliant member define a third angle therebetween. The third angle is less than 180° when the compliant member is in its second position.

In embodiments, the compliant member is the only physical link between the movable handle and the drive assembly.

It is additionally disclosed that the driver connector is “L”-shaped and includes a first leg and a second leg. The first leg is pivotably engaged with the proximal driver, and the distal driver is pivotably engaged with the driver connector at an intersection between the first leg and the second leg.

In disclosed embodiments, actuation of the pivotable handle causes distal movement of the proximal driver, which causes the driver connector to pivot from a first position where the driver connector is in contact with a pusher pin of the pusher assembly, to a second position where the driver connector is free from contact with the pusher pin. Additionally, it is disclosed that the pusher pin travels within a slot of the elongated portion. A proximal portion of the slot and a distal portion of the slot are disposed at an angle relative to the longitudinal axis, and an intermediate portion of the slot is parallel to the longitudinal axis.

It is further disclosed that the actuation of the pivotable handle causes distal movement of the distal driver to form a surgical clip.

Further details and aspects of exemplary embodiments of the disclosure are described in more detail below with reference to the appended figures.

### BRIEF DESCRIPTION OF THE FIGURES

Embodiments of the disclosure are described herein with reference to the accompanying drawings, wherein:

FIG. 1 is a perspective view of a surgical clip applier in accordance with an embodiment of the disclosure;

FIG. 2 is a schematic view of various components of a handle assembly of the surgical clip applier of FIG. 1, shown in a first, unactuated position;

FIG. 3 is an enlarged view of the area of detail indicated in FIG. 2 illustrating the connection between a pivotal handle and a compliant member of the handle assembly;

FIG. 4 is a schematic view illustrating the engagement between the pivotal handle and the compliant member of the handle assembly of the surgical clip applier of FIG. 1;

FIG. 5 is a schematic view of various components of the handle assembly of the surgical clip applier of FIG. 1, shown in a second, actuation position;

FIG. 6 is an enlarged view of the area of detail indicated in FIG. 1 illustrating a distal end of the surgical clip applier with a surgical clip therein;

FIG. 7 is a perspective view of a distal portion of the surgical clip applier of FIG. 1 with portions omitted, illustrating a plurality of surgical clips and a distal portion of a pusher assembly;

FIG. 8 is a perspective view of a pusher assembly of the surgical clip applier of FIG. 1;

FIG. 9 is a perspective view of the distal portion of the surgical clip applier of FIG. 1 with portions omitted, illustrating a surgical clip, jaw members, and a portion of a drive assembly;

FIG. 10 is a cross-sectional view of a distal portion of the surgical clip applier taken along line 10-10 in FIG. 1;

FIG. 11 is a cross-sectional view of a distal portion of the surgical clip applier taken along line 11-11 in FIG. 1;

FIG. 12 is an enlarged view of the area of detail indicated in FIG. 10 illustrating the drive assembly in an initial position;

FIG. 13 is a schematic view of a portion of the drive assembly of the surgical clip applier of FIG. 1 illustrated in a partially-actuated position;

FIG. 14 is a cross-sectional view of the distal portion of the surgical clip applier of FIG. 1 corresponding to the drive assembly being in a partially-actuated position;

FIG. 15 is a schematic view of a portion of the drive assembly of the surgical clip applier of FIG. 1 showing the pusher assembly in a distal-most position;

FIG. 16 is a cross-sectional view of the distal portion of the surgical clip applier of FIG. 1 corresponding to the pusher assembly being in the distal-most position of FIG. 15;

FIG. 17 is a cross-sectional view of the distal portion of the surgical clip applier of FIG. 1 corresponding to a distal driver of the drive assembly being in a distal-most position;

FIG. 18 is a schematic view of a portion of the drive assembly of the surgical clip applier of FIG. 1 illustrated during the start of retraction;

FIG. 19 is a schematic view of a portion of the drive assembly of the surgical clip applier of FIG. 1 illustrated in a partially-retracted position; and

FIG. 20 is a schematic view of a portion of the drive assembly of the surgical clip applier of FIG. 1 illustrated in a fully-retracted position.

### DETAILED DESCRIPTION

Embodiments of the disclosed surgical instrument are described in detail with reference to the drawings, in which like reference numerals designate identical or corresponding elements in each of the several views. Non-limiting examples of surgical instruments and associated handle assemblies according to the disclosure include manual, robotic, mechanical and/or electromechanical surgical staplers, forceps, tack appliers (e.g., tackers), clip appliers, and the like. As used herein the term “distal” refers to that portion of the surgical instrument, or component thereof, farther from the user, while the term “proximal” refers to that portion of the surgical instrument, or component thereof, closer to the user.

As will be described in greater detail below, the disclosure includes a surgical instrument, such as an endoscopic surgical clip applier, having a simplified handle assembly and a simplified actuation assembly. The handle assembly and the drive assembly are configured to advance and form a surgical clip, for instance.

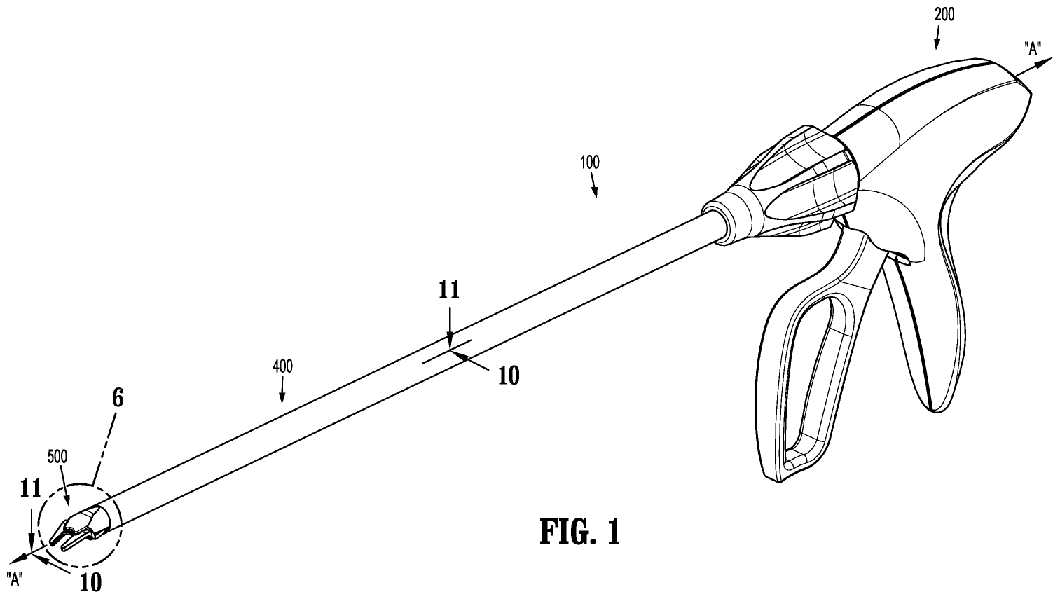

FIGS. 1-20 illustrate an endoscopic surgical clip applier in accordance with an embodiment of the disclosure generally referred to as a surgical instrument 100. The surgical instrument includes a handle assembly 200, an endoscopic or elongated portion 400 extending distally from the handle assembly 200 and defining a longitudinal axis “A-A,” and an end effector 500 disposed adjacent a distal end of the elongated portion 400. Additionally, a drive assembly 300 is disposed in mechanically cooperation with the handle assembly 200 and is configured to distally advance and form surgical clips 600.

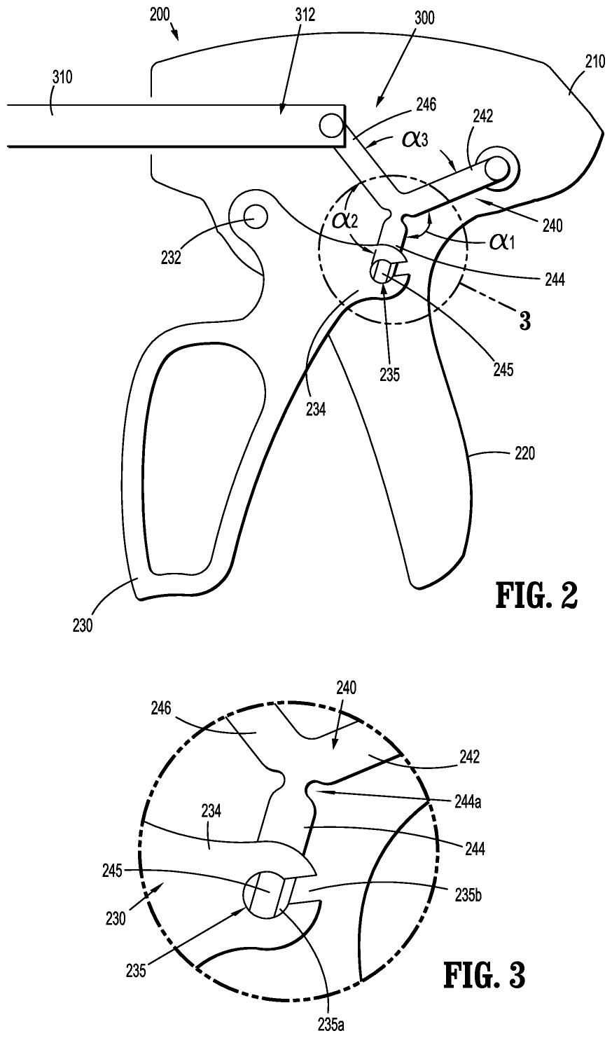

With particular reference to FIG. 2, the handle assembly 200 includes a handle housing 210, a stationary handle 220, a pivotable handle 230, and a compliant member 240. Additionally, a proximal portion 312 of a proximal driver 310 of the drive assembly 300 is shown within the handle housing 210 of the handle assembly 200. The pivotable handle 230 is pivotable relative to the handle housing 210 about a handle pivot pin 232.

Referring now to FIGS. 2-5, the compliant member 240 interconnects the handle housing 210, the pivotable handle 230, and the proximal portion 312 of the proximal driver 310 of the drive assembly 300. More particularly, the compliant member 240 is a unitary structure that can be made from polymers (e.g., injection molded, machined, additively manufactured), composite materials, shape memory alloys, elastomers, or 3D-printed metals, for instance. More particularly, an injected molded compliant member 240 may be made from polypropylene or polyethylene, for example. A compliant member 240 made with composite materials can be made by overmolding plastics onto a spring steel frame, which would be sufficient to provided desired rigidity and flexibility.

The compliant member includes a first arm 242, a second arm 244, and a third arm 246. The first arm 242 is configured to pivotably engage the handle housing 210, the second arm 244 is configured to pivotably engage a wing 234 of the pivotable handle 230, and the third arm 246 is configured to pivotably engage the proximal portion 312 of the proximal driver 310 of the drive assembly 300.

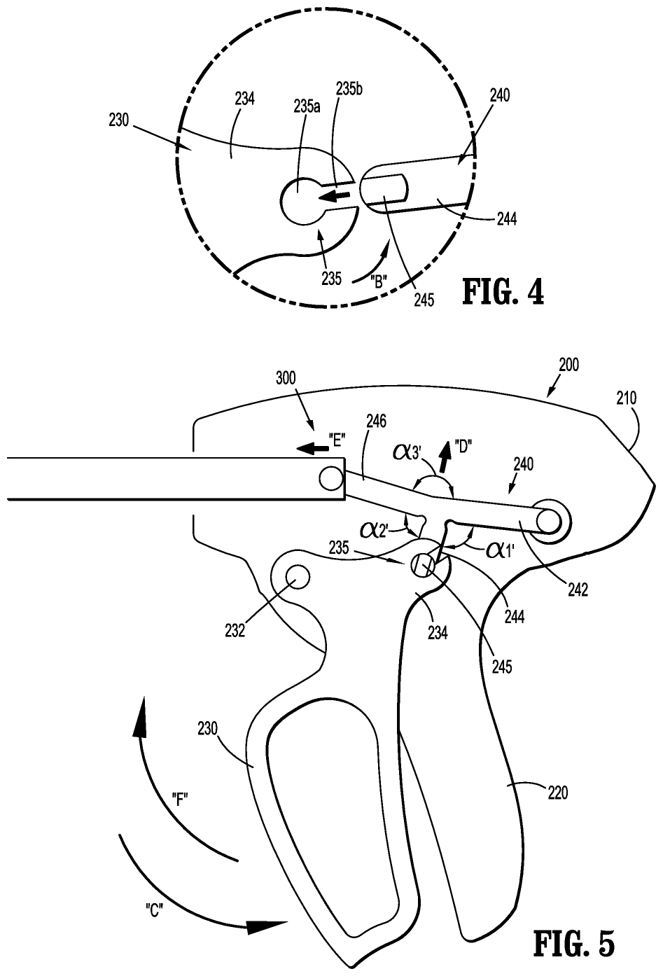

The engagement between the second arm 244 of the compliant member 240 and the wing 234 of the pivotable handle 230 is shown in FIGS. 3 and 4. The wing 234 of the pivotable handle 230 includes keyhole-shaped opening 235 having a circular portion 235a and a rectangular portion 235b. The second arm 244 of the compliant member 240 includes a finger 245 configured to be inserted into the keyhole-shaped opening 235 of the wing 234 of the pivotable handle 230 (FIG. 4). After this insertion, the second arm 244 is rotated in the general direction of arrow “B” (FIG. 4) relative to the pivotable handle 230 such that the finger 245 of the second arm 244 is retained within the circular portion 235a of the keyhole-shaped opening 235 (FIG. 3).

With particular reference to FIG. 3, the second arm 244 also includes a relatively narrow portion 244a near its intersection with the first arm 242 and the third arm 246. The narrow portion 244a may facilitate the movement of the compliant member 240 during actuation of the pivotable handle 230, as described below.

Referring back to FIG. 2, the pivotable handle 230 is shown in its unactuated position. Here, the compliant member 240 forms three angles: a first angle “α1” between the first arm 242 and the second arm 244; a second angle “α2” between the second arm 244 and the third arm 246; and third angle “α3” between the first arm 242 and the third arm 244. In the illustrated orientation, each of the angles “α1, α2 and α3” are obtuse, and are between about 100° and 140° (e.g., approximately equal to 120°). In this position, the proximal driver 310 is in its proximal-most position.

With particular reference to FIG. 5, the pivotable handle 230 is shown in its actuated position after being pivoted about the handle pivot pin 232 relative to the handle housing 210 in the general direction of arrow “C.” Here, the movement of the wing 234 of the pivotable handle 230 forces the second arm 244 of the compliant member 240 in the general direction of arrow “D.” As the second arm 244 moves in the general direction of arrow “D,” the third arm 246, and thus the proximal driver 310, move distally in the general direction of arrow “E.” When the compliant member 240 is in this position, the first angle “α1′” and the second angle “α2′” are smaller, and the third angle “α3′” is larger compared to the corresponding angles “α1, α2 and α3” when the pivotable handle 230 is in the unactuated position (FIG. 2). In the actuated position of FIG. 5, the first angle “α1′” and the second angle “α2′” are between about 91° and about 100° (e.g., about 95°), and the third angle “α3′” is less than 180°, and is between about 160° and about 179° (e.g., about 170°). The fact that the third angle “α3′” remains less than 180° helps ensure the compliant member 240 does not bind in an undesired position.

After actuation of the pivotable handle 230, the pivotable handle 230 is moved in the general direction of arrow “F” in FIG. 5 back to its pre-actuated position shown in FIG. 2. This movement of the pivotable handle 230 causes the compliant member 240 to return to its pre-actuated position (FIG. 2), and causes the proximal driver 310 to move proximally to its pre-actuated position (FIG. 2).

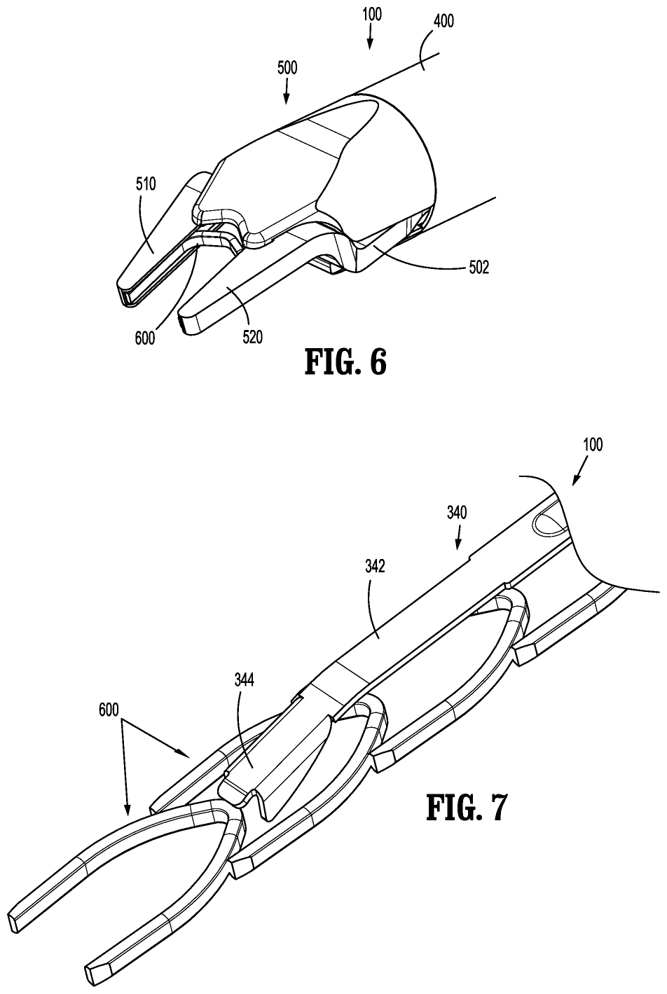

Referring now to FIGS. 6-20, further details of the drive assembly 300 and the distal portion of the surgical instrument 100 are shown. The drive assembly 300 includes a proximal driver 310, a distal driver 320, a driver connector 330, and a pusher assembly 340. Generally, actuation of the pivotable handle 230 causes the proximal driver 310, the distal driver 320, and the pusher assembly 340 to move distally, which in turn, causes distal advancement of a surgical clip 600 from within the elongated portion 400 and formation of the surgical clip 600.

As shown in FIGS. 6, 7 and 9, a plurality of surgical clips 600 is disposed adjacent the distal portion of the surgical instrument 100. More particularly, the end effector 500 includes a first jaw member 510 and a second jaw member 520. The pusher assembly 340 of the drive assembly 300 advances surgical clips 600, one at a time, to a position between the first jaw member 510 and the second jaw member 520, and the distal driver 320 of the drive assembly 300 is advanced to approximate the first jaw member 510 and the second jaw member 520, which compresses and forms the surgical clip 600 (e.g., through tissue). After the distal-most surgical clip 600 is formed (and ejected), subsequent actuation of the pivotable handle 230 results in distal advancement and formation of the next distal-most surgical clip 600.

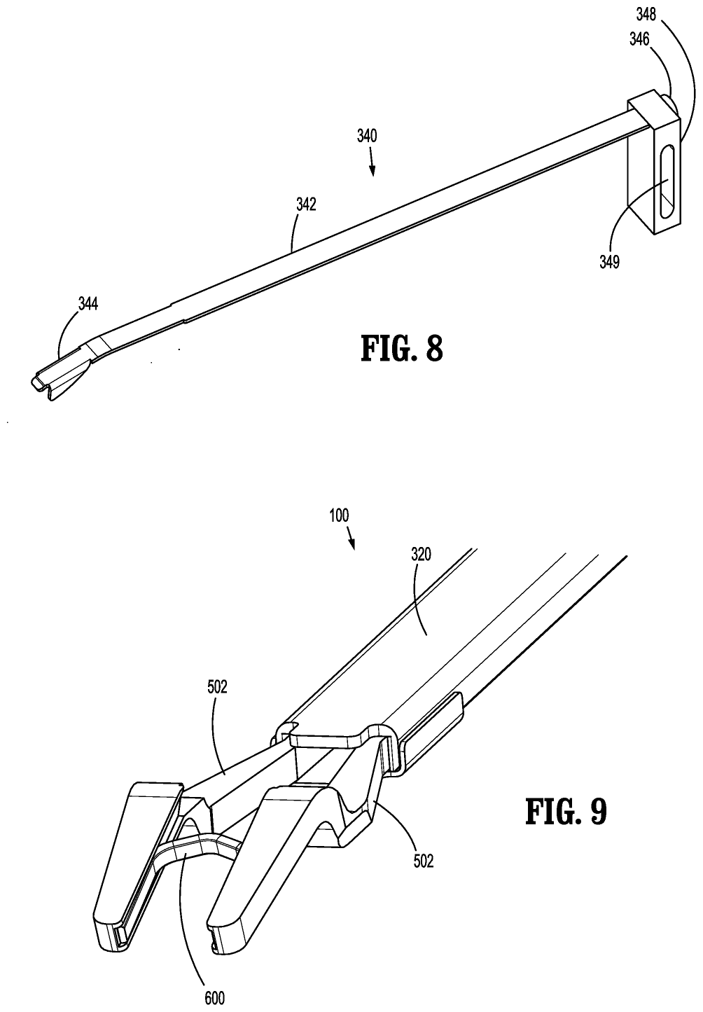

With particular reference to FIG. 8, the pusher assembly 340 is shown. The pusher assembly 340 includes a pusher rod 342, an engagement portion 344, a boss 346, and a finger 348. The engagement portion 344 is disposed at a distal end of the pusher rod 342 and is configured to contact the distal-most surgical clip 600. The boss 346 is disposed at a proximal end of the pusher rod 342 and is configured to accept a biasing element 350 (see FIG. 12, for example). The finger 348 depends from a proximal portion of the pusher rod 342 and includes a slot 349 configured to accept a pusher pin 360 (see FIG. 12, for example). The finger 348 is fixed from movement relative to the pusher rod 342.

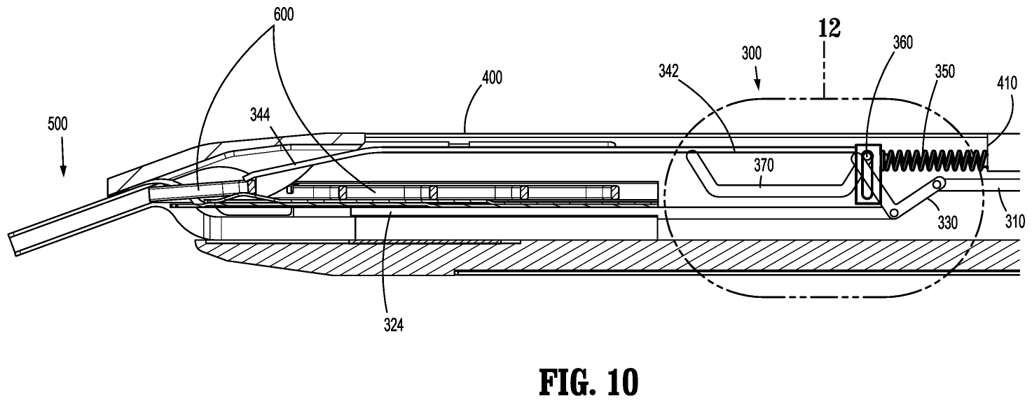

Referring now to FIGS. 10-20, further details of the drive assembly 300 are shown. As shown in FIG. 12, for instance, the driver connector 330 is generally “L”-shaped and includes a first leg 332, a second leg 334, a first pivot location 336 on the first leg 332, and a second pivot location 338 disposed at or near the intersection of the first leg 332 and the second leg 334. The first leg 332 is pivotably connected to a distal end 314 of the proximal driver 310 at the first pivot location 336. The driver connector 330 is also pivotably connected to a proximal end 322 of the distal driver 320 at the second pivot location 338 of the driver connector 330.

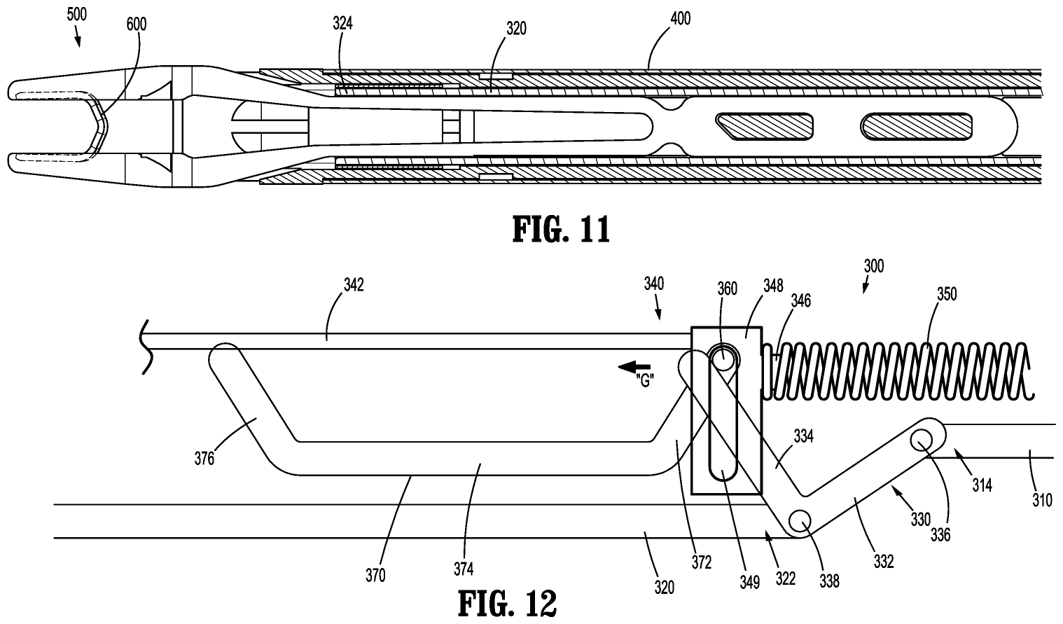

With particular reference to FIGS. 10-12, the drive assembly 300 is in an initial position corresponding to when the pivotable handle 230 of the handle assembly 200 has not been actuated (as in FIGS. 1 and 2). In this position, the biasing element 350 (e.g., compression spring) is shown between a wall 410 (FIG. 10) of the elongated portion 400 and the boss 346 of the pusher assembly 340, and biases the pusher assembly 340 distally in the general direction of arrow “G” in FIG. 12. The second leg 334 of the driver connector 330 is in contact with the pusher pin 360, opposing the distal force created by the biasing element 350, thereby preventing distally translation of the pusher pin 360 and the pusher assembly 340. As shown in FIG. 11, in this position, a distal end 324 of the distal driver 320 is positioned proximally of the jaw members 500.

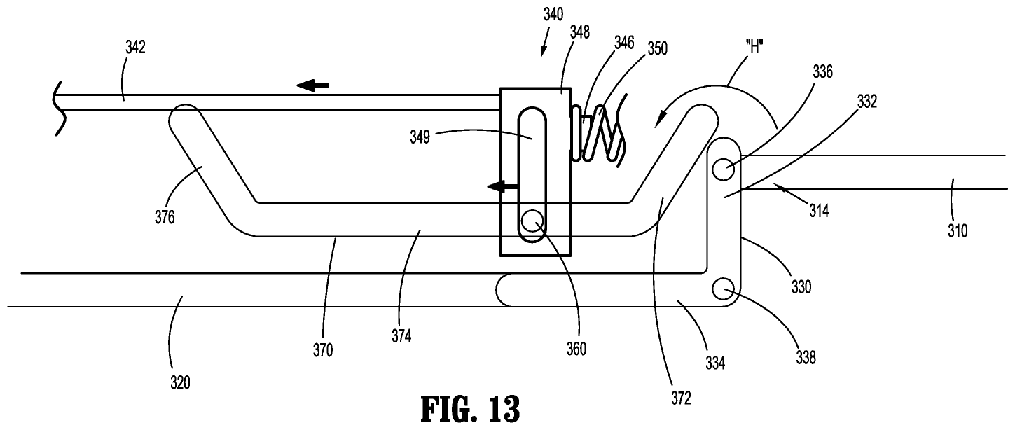

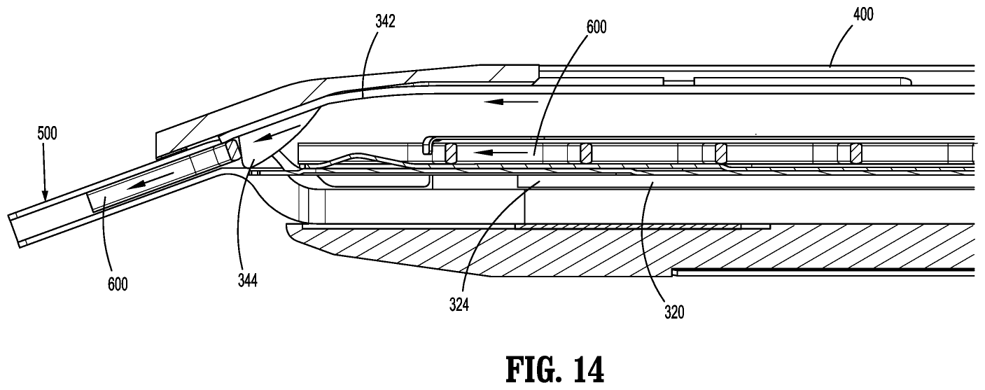

Referring now to FIGS. 13 and 14, the drive assembly 300 is in a partially-actuated position corresponding to when the pivotable handle 230 of the handle assembly 200 has been partially actuated, but is not yet in the fully actuated position shown in FIG. 5. Here, the distal movement of the proximal driver 310 (in response to a partial actuation of the pivotable handle 230) causes the driver connector 330 to pivot about the first pivot location 336 and about the second pivot location 338 in the general direction of arrow “H” in FIG. 13. This pivotal movement of the driver connector 330 causes the second leg 334 of the driver connector 330 to move out of engagement with the pusher pin 360. Consequently, the distal force provided by the biasing element 350 is no longer opposed, and the pusher assembly 340 moves distally. More particularly, the pusher pin 360 moves within the slot 349 of the finger 348 of the pusher assembly 340, and also moves along a path or channel 370 within the elongated portion 400. The path 370 is generally “U”-shaped and includes an angled proximal portion 372, a linear middle portion 374 and an angled distal portion 376. The angles of the proximal portion 372 and the distal portion 376 relative to the middle portion 374 may be between about 30° and about 60° (e.g., equal to about 45°).

As shown in FIG. 14, the pusher rod 342, in response to the force exerted by the biasing element 350, moves distally toward its distal-most position. This distal movement of the pusher rod 342 results in the engagement portion 344 at the distal end of the pusher rod 342 forcing a surgical clip 600 generally distally toward its distal-most position between the jaw members of the end effector 500. In this position, the distal end 324 of the distal driver 320 remains positioned proximally of the jaw members 500.

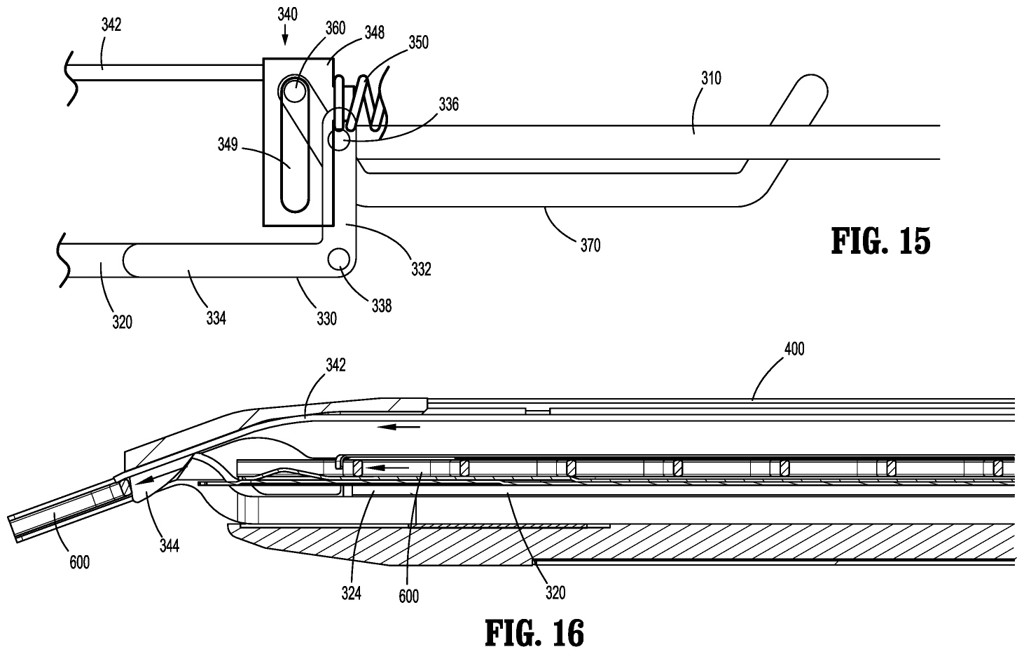

With reference to FIGS. 15 and 16, the pusher rod 342 is in its distal-most position, and the distal driver 320 is approaching its distal-most position. As shown in FIG. 16, the distal end 324 of the distal driver 320 is positioned proximally of the end effector 500. In particular, the distal end 324 of the distal driver 320 is positioned proximally of camming surfaces 502 (shown in FIGS. 6, 9 and 17) of the jaw members of the end effector 500. Accordingly, a partial actuation of the pivotable handle 230 causes the distal-most surgical clip 600 to be advanced distally prior to the distal driver 320 reaching its distal-most position.

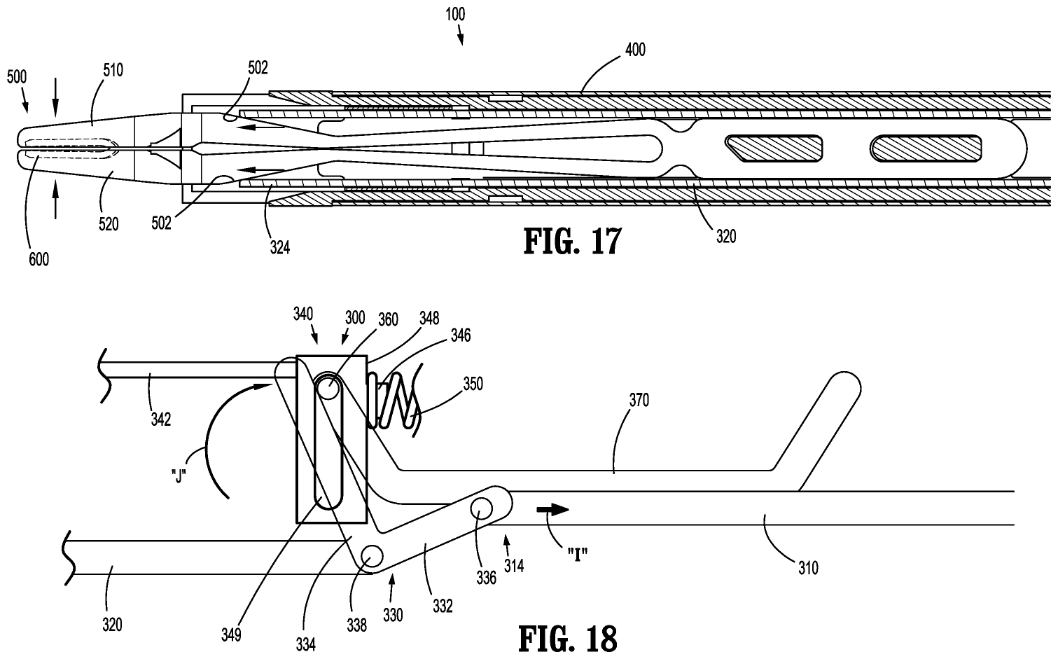

Referring to FIG. 17, the distal end of the surgical instrument 100 is shown when the distal driver 320 is in its distal-most position. Here, the distal end 324 of the distal driver 320 is engaged with the camming surfaces 502 of the end effector 500, which caused the first jaw member 510 and the second jaw member 520 to move to their approximated position, thereby compressing and forming the surgical clip 600.

With reference to FIGS. 18-20, following the formation of the surgical clip 600, the drive assembly 300 is retracted. As discussed above, returning the pivotable handle 230 toward its initial, pre-actuated position, causes the proximal driver 310 to move proximally, or retract. The proximal movement of the proximal driver 310 in the general direction of arrow “I” in FIG. 18, causes the driver connector 330 to pivot about the first pivot location 336 and about the second pivot location 338 in the general direction of arrow “J” in FIG. 18. This pivotal movement of the driver connector 330 causes the second leg 334 of the driver connector 330 to move into engagement with or contact the pusher pin 360. The second leg 334 of the driver connector 330 may include a lip or other engagement structure to facilitate the engagement with the pusher pin 360.

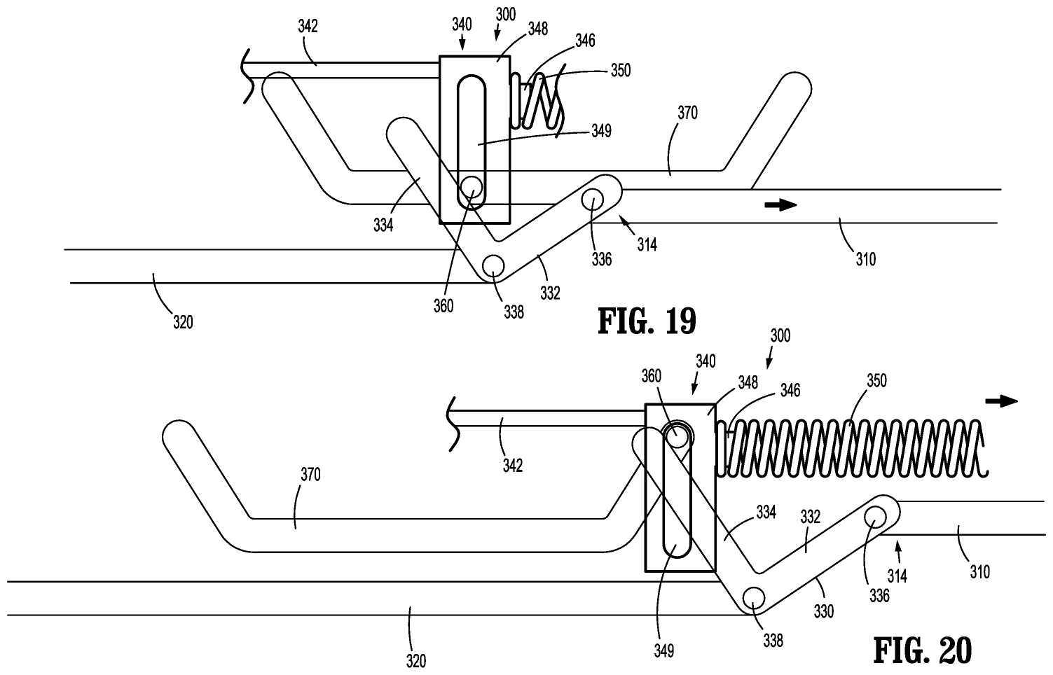

As shown in FIG. 19, continued proximal movement of the proximal driver 320 causes the driver connector 300 and the distal driver 330 to move proximally. The engagement between the driver connector 330 and the pusher pin 360 results in the pusher pin 360 moving generally proximally with the path 370 in response to the proximal movement of the driver connector 330. As the pusher pin 360 moves generally proximally, the pusher pin 360 also moves within the slot 349 of the finger 348 (FIG. 20). In response, the finger 348 and the pusher rod 342 also move proximally, thereby causing the biasing element 350 to compress. FIG. 20 shows the drive assembly 300 in its proximal-most position.

It should be understood that the foregoing description is only illustrative of the disclosure. Various alternatives and modifications can be devised by those skilled in the art without departing from the disclosure. Accordingly, the disclosure is intended to embrace all such alternatives, modifications, and variances. The embodiments described with reference to the accompanying figures are presented only to demonstrate certain examples of the disclosure. Other elements, steps, methods, and techniques that are insubstantially different from those described above and/or in the appended claims are also intended to be within the scope of the disclosure.

What is claimed:

1. An actuation mechanism for use with a surgical instrument having a housing, a pivotable handle, and a driver, the actuation mechanism comprising:
a compliant member including a first arm, a second arm, and a third arm, the first arm including a first pivot point configured to pivotably engage the housing of the surgical instrument, the second arm including a second pivot point configured to pivotably engage the pivotable handle of the surgical instrument, and the third arm including a third pivot point configured to pivotably engage the driver of the surgical instrument,
wherein the compliant member is a unitary structure, and is movable between a first position and a second position in response to the pivotable handle moving from a first, unactuated position to a second, actuated position.

2. The actuation mechanism according to claim 1, wherein the first arm and the second arm of the compliant member define a first angle therebetween, the second arm and the third arm of the compliant member define a second angle therebetween, the first arm and the third arm of the compliant member define a third angle therebetween, and the third angle is less than 180° when the compliant member is in its second position.

3. The actuation mechanism according to claim 2, wherein the third angle is less than 180° when the compliant member is in its first position.

4. The actuation mechanism according to claim 3, wherein the first angle and the second angle are obtuse when the compliant member is in the first position.

5. The actuation mechanism according to claim 4, wherein the first angle and the second angle are obtuse when the compliant member is in the second position.

6. The actuation mechanism according to claim 1, wherein the compliant member is made from one of a polymer, a composite material, a shape memory alloy, an elastomers, or a 3D-printed metal.

7. A drive assembly for use with a surgical instrument, comprising:
a proximal driver disposed in mechanical cooperation with a handle assembly of the surgical instrument;
a driver connector having a first leg and a second leg, the first leg is pivotably engaged with the proximal driver;
a distal driver pivotably engaged with the driver connector and configured to form a surgical fastener; and
a pusher assembly including a biasing element, a pusher pin, and a pusher rod, the biasing element biasing the pusher rod distally, wherein distal movement of the pusher rod is configured to distally advance a surgical fastener;
wherein the driver connector is movable between a first position where the driver connector is in contact with the pusher pin and prevents distal movement of the pusher pin, and a second position where the driver connector is free from contact with the pusher pin, thereby allowing distal movement of the pusher pin.

8. The drive assembly according to claim 7, further including a slot, wherein the pusher pin is configured to travel within the slot.

9. The drive assembly according to claim 8, wherein the slot is generally “U”-shaped.

10. The drive assembly according to claim 7, wherein the distal driver is pivotably engaged with the driver connector at an intersection between the first leg and the second leg.

11. The drive assembly according to claim 7, wherein distal movement of the proximal driver causes the driver connector to move from its first position to its second position.

12. The drive assembly according to claim 11, wherein proximal movement of the proximal driver causes the driver connector to move from its second position to its first position.

13. A surgical clip applier, comprising:
a handle assembly including a housing, a pivotable handle, and a stationary handle;
an elongated portion extending distally from the handle assembly and defining a longitudinal axis;
an end effector disposed adjacent a distal end of the end effector and including a first jaw member and a second jaw member;
a drive assembly disposed at least partially within the elongated portion and including a proximal driver, a driver connector, a distal driver, and a pusher assembly, the driver connector pivotably engaged with the proximal driver and with the distal driver, the distal driver configured to engage a portion of the end effector, the pusher assembly configured to distally advance a surgical clip; and
a compliant member disposed at least partially within the housing and including a first arm, a second arm, and a third arm, the first arm including a first pivot point configured to pivotably engage the housing of the handle assembly, the second arm including a second pivot point configured to pivotably engage the pivotable handle of the handle assembly, and the third arm including a third pivot point configured to pivotably engage the proximal driver of the drive assembly.

14. The surgical clip applier according to claim 13, wherein the compliant member is a unitary structure, and is movable between a first position and a second position in response to the pivotable handle moving from a first, unactuated position to a second, actuated position.

15. The surgical clip applier according to claim 14, wherein the first arm and the second arm of the compliant member define a first angle therebetween, the second arm and the third arm of the compliant member define a second angle therebetween, and the first arm and the third arm of the compliant member define a third angle therebetween, and wherein the third angle is less than 180° when the compliant member is in its second position.

16. The surgical clip applier according to claim 13, wherein the compliant member is the only physical link between the pivotable handle and the drive assembly.

17. The surgical clip applier according to claim 13, wherein the driver connector is “L”-shaped and includes a first leg and a second leg, the first leg is pivotably engaged with the proximal driver, and the distal driver is pivotably engaged with the driver connector at an intersection between the first leg and the second leg.

18. The surgical clip applier according to claim 13, wherein actuation of the pivotable handle causes distal movement of the proximal driver, which causes the driver connector to pivot from a first position where the driver connector is in contact with a pusher pin of the pusher assembly, to a second position where the driver connector is free from contact with the pusher pin.

19. The surgical clip applier according to claim 18, wherein the pusher pin travels within a slot of the elongated portion, a proximal portion of the slot and a distal portion of the slot are disposed at an angle relative to the longitudinal axis, and an intermediate portion of the slot is parallel to the longitudinal axis.

20. The surgical clip applier according to claim 18, wherein the actuation of the pivotable handle causes distal movement of the distal driver to form a surgical clip.
